(19) United States
(12) Patent Application Publication
(10) Pub. No.: US 2026/0256609 A1
(43) Pub. Date: Sep. 3, 2026

(54) ORTHOSIS SUPPORT FRAME

(71) Applicant: Meredith Phillips, Bethlehem, PA (US)

(72) Inventor: Meredith Phillips, Bethlehem, PA (US)

(21) Appl. No.: 19/067,413

(22) Filed: Feb. 28, 2025

(51) Int. Cl. A61F 5/01

(52) U.S. Cl. CPC A61F 5/0127

(57) ABSTRACT

A brace support frame may comprise a base member having an interior surface and an exterior surface. At least a portion of the interior surface of the base member may include an interior surface feature. At least a portion of the exterior surface of the base member may include an exterior surface feature. The brace support frame may include a first sidewall member having an interior surface and an exterior surface. At least a portion of the exterior surface of the first sidewall member may include one or more first attachment features. The brace support frame may include a second sidewall member having an interior surface and an exterior surface. At least a portion of the exterior surface of the second sidewall member may include one or more second attachment features. The brace support frame may include an endwall member having an interior surface and an exterior surface.

### BACKGROUND OF THE INVENTION

### Field of the Invention

Disclosed herein are devices useful with orthoses. Specifically, disclosed herein are devices useful for the fitment of orthoses by individuals having limited mobility or flexibility.

### Description of Related Art

An orthosis provides support and stability to a joint and can provide a multitude of benefits such as: reducing pain and/or swelling in the joint, preventing further damage or deterioration of the joint, or aiding in the healing of an injury to the joint. A joint brace functions by providing a compressive force to the joint and limiting excess movement or displacement of the joint. Typically, such braces are fabricated using a strong, flexible material that is secured about the joint or body part using fasteners such as laces, buckles, or hook-and-loop fasteners.

FIG. 1 depicts an illustrative orthosis in the form of a brace 100 to provide support to a joint such as a wrist, elbow, knee, or ankle. Although described in terms of an ankle support brace 100 in the following discussion, it should be understood the apparatuses, systems, and methods described herein find equal applicability to other apparatuses that assist in installing, affixing or donning support braces on other joints or extremities such as wrist support braces, knee support braces, and elbow support braces.

As depicted in FIG. 1, the ankle support brace 100 includes a central section 102 flanked on a first side by first attachment devices 104A-104n (collectively, “first attachment devices 104”) and across the width of the central section 102 by a corresponding number of second attachment devices 106A-106n (collectively, “second attachment devices 106”), in accordance with one or more embodiments described herein. Each of the first attachment devices 104 includes an attachment device or feature 108A-108n (collectively, “attachment features 108”). Example attachment features 108 include but are not limited to: hook-and-loop fasteners, buttons, snaps, lace eyelets, buckles, a snap fit, or similar devices. Each of the second attachment devices 106 includes an attachment device or feature 110A-110n (collectively, “attachment features 110”) each of which is complimentary to the attachment feature 108 on corresponding ones of the first attachment devices 104. Each of the attachment features 108 can be integral with or attached, affixed, or otherwise bonded to each of the respective first attachment devices 104. Each of the attachment features 110 can be integral with or attached, affixed, or otherwise bonded to each of the respective first attachment devices 106.

It should be noted that although the illustrative brace depicted in FIG. 1 includes a number of first attachment devices 104 equal to the number of second attachment features 106, the number of first attachment features 104 can be lesser or greater than the number of second attachment features 106. For example, two first attachment devices (e.g., 104A and 104B) may attach to a single, common, second attachment device (e.g., 106A)-such embodiments should be considered as included within the scope of this disclosure

The ankle support brace 100 includes a lower ankle brace portion 120 that, when installed, extends around or surrounds the wearer's foot and an upper ankle brace portion 130 that, when installed, extends around or surrounds the wearer's ankle and lower calf. The ankle support brace 100 provides support to the ankle joint, improving the wearer's mobility and sense of comfort. To attach the ankle support brace 100, each of the first attachment devices 104 and each of the second attachment devices 106 are extended at least partially over a portion of either the foot, ankle, or lower calf such that the attachment features 108 disposed, formed, in, or carried by, each of the first attachment devices 104 engage at least one of the attachment features 110 disposed, formed, in, or carried by, each of the second attachment devices 106. In the example ankle support brace 100 depicted in FIG. 1, the one or more first attachment devices 104 and the one or more second attachment devices 106 may include hook-and-loop type fasteners that are used to attach the ankle brace about the foot, ankle, and lower portion of the wearer's calf.

While donning the ankle support brace 100 is relatively straightforward, the first attachment devices 104 with attachment features 108 and/or the second attachment devices 106 with attachment features 110 may sometimes be displaced, stick together, become entangled, thereby increasing the difficulty in correctly installing the ankle support brace 100. For elderly patients and patients having limited mobility, entanglement or adhesion of the first attachment devices 104 and/or the second attachment devices 106 can present a significant challenge that impedes or interferes with the installation of the ankle support brace 100.

### BRIEF DESCRIPTION OF THE DRAWINGS

FIG. 1 depicts an illustrative joint support brace, such as an ankle support brace, in accordance with one or more embodiments described herein.

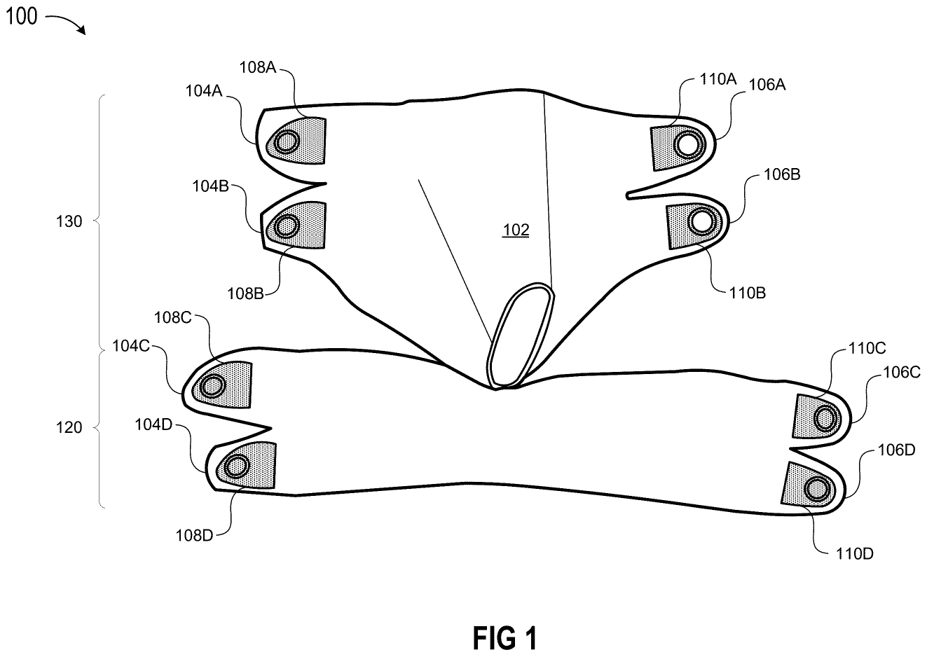

FIG. 2 is a perspective view of an illustrative ankle support brace frame in accordance with one or more embodiments described herein.

FIG. 3 is a perspective view of another illustrative ankle support brace frame in accordance with one or more embodiments described herein.

FIG. 4A is a top plan view of the illustrative ankle support brace frame as depicted in FIG. 2, in accordance with one or more embodiments described herein.

FIG. 4B is a top plan view of another illustrative embodiment of the ankle support brace frame depicted in FIG. 2 in which one or more hinges rotatably couple the sidewall members to the base member and one or more hinges rotatably couple the endwall member to the base member, in accordance with one or more embodiments described herein.

FIG. 4C is a top plan view of yet another illustrative embodiment of the ankle support brace frame depicted in FIG. 2 in which one or more hinges rotatably couple each of the sidewall members to the endwall member, in accordance with one or more embodiments described herein.

FIG. 5A is a bottom plan view of the ankle support brace frame as depicted in FIG. 2, in accordance with one or more embodiments described herein.

FIG. 5B is a bottom plan view of the illustrative embodiment of the ankle support brace frame depicted in FIG. 4B in which one or more hinges rotatably couple the sidewall members to the base member and one or more hinges rotatably couple the endwall member to the base member, in accordance with one or more embodiments described herein.

FIG. 5C is a bottom plan view of the illustrative embodiment of the ankle support brace frame depicted in FIG. 4C in which one or more hinges rotatably couple each of the sidewall members to the endwall member, in accordance with one or more embodiments described herein.

FIG. 6A is a side elevation view of the ankle support brace frame as depicted in FIG. 2, in accordance with one or more embodiments described herein.

FIG. 6B is a side elevation view of another embodiment of the ankle support brace frame depicted in FIG. 2 in which the sidewall and endwall members are rotatably coupled to the base member via one or more hinges, in accordance with one or more embodiments described herein.

FIG. 6C is a side elevation view of another embodiment of the ankle support brace frame depicted in FIG. 2 in which the sidewall members are rotatably coupled to the endwall member via one or more hinges, in accordance with one or more embodiments described herein.

FIG. 7A is an open-end elevation view of the ankle support brace frame as depicted in FIG. 2, in accordance with one or more embodiments described herein.

FIG. 7B is an open-end elevation view of another embodiment of the ankle support brace frame depicted in FIG. 2 in which the sidewall and endwall members are rotatably coupled to the base member via one or more hinges, in accordance with one or more embodiments described herein.

FIG. 7C is an open-end elevation view of yet another embodiment of the ankle support brace frame depicted in FIG. 2 in which the sidewall members are rotatably coupled to the endwall member via one or more hinges, in accordance with one or more embodiments described herein.

FIG. 8A is a closed-end elevation view of the ankle support brace frame as depicted in FIG. 2, in accordance with one or more embodiments described herein.

FIG. 8B is a closed-end elevation view of another embodiment of the ankle support brace frame depicted in FIG. 2 in which the sidewall and endwall members are rotatably coupled to the base member via one or more hinges, in accordance with one or more embodiments described herein.

FIG. 8C is a closed-end elevation of yet another embodiment of the ankle support brace frame depicted in FIG. 2 in which the sidewall members are rotatably coupled to the endwall member via one or more hinges, in accordance with one or more embodiments described herein.

FIG. 9A is a perspective view of an illustrative ankle support brace in an opened position disposed at least partially in the ankle support brace frame depicted in FIG. 2, in accordance with one or more embodiments described herein.

FIG. 9B is a perspective view of the illustrative ankle support brace in the opened position disposed at least partially in the ankle support brace frame depicted in FIG. 2, with a wearer's foot positioned within the opened ankle support brace, in accordance with one or more embodiments described herein.

FIG. 9C is a perspective view of the illustrative ankle support brace in a closed or fitted position about the wearer's foot while the wearer's foot and the illustrative ankle support brace are positioned in the ankle support brace frame depicted in FIG. 2, in accordance with one or more embodiments described herein.

FIG. 10 is a perspective view of yet another embodiment of an ankle support brace frame in accordance with one or more embodiments described herein.

FIG. 11 is a top plan view of the ankle support brace frame depicted in FIG. 10, in accordance with one or more embodiments described herein.

FIG. 12 is a bottom plan view of the ankle support brace frame depicted in FIG. 10, in accordance with one or more embodiments described herein.

FIG. 13 is a side elevation of the ankle support brace frame depicted in FIG. 10, in accordance with one or more embodiments described herein.

FIG. 14 is an open-end elevation view of the ankle support brace frame depicted in FIG. 10, in accordance with one or more embodiments described herein.

FIG. 15 is a closed-end elevation view of the ankle support brace frame depicted in FIG. 10, in accordance with one or more embodiments described herein.

FIG. 16A is a perspective view of an illustrative ankle support brace in an opened position disposed at least partially in the ankle support brace frame depicted in FIG. 10, in accordance with one or more embodiments described herein.

FIG. 16B is a perspective view of the illustrative ankle support brace in the opened position disposed at least partially in the ankle support brace frame depicted in FIG. 10, with a wearer's foot positioned within the opened ankle support brace, in accordance with one or more embodiments described herein.

FIG. 16C is a perspective view of the illustrative ankle support brace in a closed or fitted position about the wearer's foot while the wearer's foot and the illustrative ankle support brace are positioned in the ankle support brace frame depicted in FIG. 10, in accordance with one or more embodiments described herein.

### DETAILED DESCRIPTION

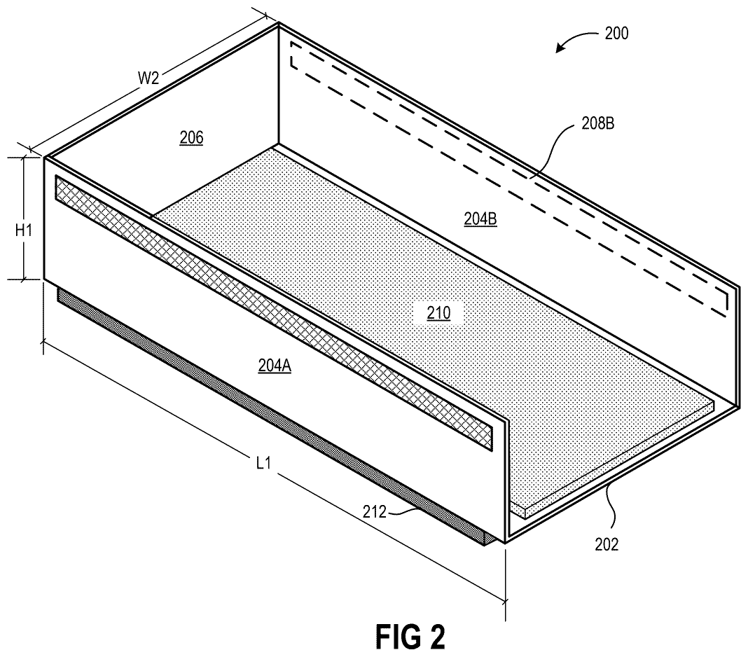

FIG. 2 is a perspective view of an illustrative ankle support brace frame 200 in accordance with one or more embodiments described herein. The ankle support brace frame 200 includes a base member 202, opposed sidewall members 204A, 204B (collectively, “sidewall members 204”), and an endwall member 206 that are joined or otherwise positionable to form an open-top, open-ended, three-sided structure that defines a volume having an interior surface and an exterior surface. The ankle support brace frame 200 can have an overall length “L1”, an overall width “W1”, and an overall height “H1” suitable to accommodate the placement of a wearer's foot on, in contact with, or proximate the base member 202 with the heel positioned proximate the endwall member 206 with the toes directed toward or extending from the open-ended portion of the ankle support brace frame 200. The ankle support brace frame 200 includes one or more attachment features 208A and 208B (collectively, “attachment features 208”) coupled, bonded, attached, or otherwise affixed to at least a portion of the exterior surface of each of the sidewall members 204A and 204B, respectively. To maintain the placement of the ankle support brace 100 within the ankle brace support frame 200 and to resist the displacement or sliding of the ankle support brace 100 within the ankle brace support frame 200, a textured, non-slip, abrasive, or similar interior surface feature 210 can be disposed in, on, across, or about at least a portion of the interior surface of the base member 202. The coefficient of friction between the ankle support brace 100 and interior surface feature 210 exceeds the coefficient of friction between the ankle support brace 100 and the bare base member 202 (i.e., the interior surface of the base member 202 in the absence of the interior surface feature 210). In embodiments, the coefficient of friction between the ankle support brace 100 and the interior surface feature can be: greater than about 0.5, greater than about 0.55 greater than about 0.6, greater than about 0.65, greater than about 0.7, or greater than about 7.5.

Similarly, to resist the movement, sliding, or displacement of the ankle support brace frame 200 on an underlying support surface, a textured, non-slip, abrasive, or similar surface feature 212 can be disposed in, on, across, or about at least a portion of the exterior surface of the base member 202. The exterior surface feature 212 increases the coefficient of friction of the exterior of the base member 202 above the coefficient of friction of the bare exterior surface of the base member 202 (i.e., the exterior surface of the base member 202 in the absence of the surface feature 212). The coefficient of friction between the exterior surface feature 212 and the underlying support surface exceeds the coefficient of friction between the bare exterior surface of the base member 202 and the underlying support surface. In embodiments, the coefficient of friction between the exterior surface feature 212 and the underlying support surface can be: greater than about 0.5, greater than about 0.55 greater than about 0.6, greater than about 0.65, greater than about 0.7, or greater than about 7.5.

In embodiments, the ankle support brace frame 200 can be a unitary structure that includes the base member 202, the sidewall members 204, and the endwall member 206. In some embodiments, the ankle support brace frame 200 can be fabricated using a metallic material such as aluminum, an aluminum alloy, or similar metal containing material. In other embodiments, the ankle support brace frame 200 can be fabricated using a non-metallic material such as cardboard, injection molded plastic, carbon fiber, or similar non-metallic materials. The height “H1,” width “W1,” and length “L1,” of the ankle support brace frame 200 can be selected to accommodate the insertion or placement of a foot and ankle at least partially within the ankle support brace frame 200 with the heel proximate, near, contacting, or a short distance from the endwall member 206. The length “L1” of the ankle support brace frame 200 can be: about 6 inches or less; about 8 inches or less; about 10 inches or less; about 12 inches or less; about 14 inches or less; or about 16 inches or less. The width “W1” of the ankle support brace frame 200 can be: about 4 inches or less; about 6 inches or less; or about 8 inches or less. The height “H1” of the ankle support brace frame 200 can be: about 3 inches or less; about 4 inches or less; about 5 inches or less; or about 6 inches or less.

The attachment features 208A and 208B are disposed in, on, across, or about at least a portion of the exterior surface of the sidewall members 204A and 204B, respectively. Attachment features 208A can include any system, device, or accessory capable of retaining at least a portion of the first attachment devices 104 and/or the attachment features 108. Similarly, attachment features 208B can include any system, device, or accessory capable of retaining at least a portion of the second attachment devices 106 and/or the attachment features 110. In some embodiments, the attachment features 208A may include a hook-and-loop type material to which some or all of the first attachment devices 104 and/or some or all of the attachment features 108 can detachably attach. Similarly, in such embodiments, attachment features 208B may include a hook-and-loop type material to which some or all of the second attachment devices 106 and/or some or all of the attachment features 110 can detachably attach.

In other embodiments, the attachment features 208 may include an elastic or resilient material coupled to the exterior surface of each of the sidewall members 204. For example, the one or more attachment features 208 can include an elastic material such as rubber, nylon, or similar elastic and/or resilient materials to hold or otherwise retain at least a portion of the first attachment devices 104 and at least a portion of the second attachment devices 106 proximate, near, or against the exterior surface of each of the sidewall members 204. In yet other embodiments, the attachment features 208 may include a mechanical fastener such as a buckle, snap, quick-release, or similar device capable of retaining, coupling, physically attaching, or otherwise holding and releasing at least a portion of the first attachment devices 104 and/or attachment features 108 and at least a portion of the second attachment devices 106 and/or attachment features 110 against the exterior surface of each of the sidewall members 204.

The interior surface feature 210 reduces the slippage, movement, or displacement of the ankle support brace 100 in the ankle brace support frame 200, particularly as the wearer slides, places, or otherwise positions their foot into the ankle support brace 100. The interior surface feature 210 provides increased friction to resist movement or displacement of the ankle support brace 100 when compared to the bare interior surface of base member 202. In embodiments, the interior surface feature 210 can include a surface treatment, finish, or preparation to increase the coefficient of friction of the interior surface of the base member 202. For example, the interior surface feature 210 can be abrasive blasted, chemically etched, or laser etched on, about, or across all or a portion of the interior surface of the base member 202. In other embodiments, the interior surface feature 210 can include one or more materials bonded, applied, adhered, laminated, or otherwise affixed to the interior surface of the base member 202 to increase the coefficient of friction over the bare interior surface of the base member 202. In embodiments, the interior surface feature 210 can be applied, distributed, or otherwise disposed evenly across all or a portion of the interior surface of the base member 202. In other embodiments, the interior surface feature 210 can be applied, distributed, or otherwise disposed in a decorative or functional pattern across all or a portion of the interior surface of the base member 202. In yet other embodiments, the interior surface feature 210 can be applied, distributed, or otherwise disposed in a random or pseudo-random pattern across all or a portion of the interior surface of the base member 202.

The exterior surface feature 212 reduces slippage of the ankle support brace frame 200 across an underlying support surface, particularly when a user inserts their foot into an ankle support brace 100 disposed in the ankle support brace frame 200. Thus, the combination of the interior surface feature 210 and the exterior surface feature 212 beneficially eases the proper fitment of the ankle support brace 100 since the brace is maintained in a stable, opened, position that is not easily displaced within the ankle support brace frame 200. The fitment of the ankle support brace 100 is further enhanced by the exterior surface feature 212 resisting the movement of the ankle support brace frame 200 across the underlying support surface as the wearer places their foot into the opened support brace positioned in the ankle support brace frame 200.

In embodiments, the exterior surface feature 212 can include a surface treatment, finish, or preparation to increase the coefficient of friction of the bare exterior surface of the base member 202. For example, the exterior surface of the base member 202 may be abrasive blasted, chemically, or laser etched on, about, or across all or a portion of the exterior surface of the base member 202. In other embodiments, the exterior surface feature 212 can include one or more elastomeric or similar materials bonded, applied, adhered, laminated, or otherwise affixed to at least a portion of the exterior surface of the base member 202. In embodiments, the exterior surface feature 212 can be applied, distributed, or otherwise disposed evenly across all or a portion of the exterior surface of the base member 202. In other embodiments, the exterior surface feature 212 can be applied, distributed, or otherwise disposed in a decorative or functional pattern across all or a portion of the exterior surface of the base member 202. In yet other embodiments, the exterior surface feature 212 can be applied, distributed, or otherwise disposed in a random or pseudo-random pattern across all or a portion of the exterior surface of the base member 202.

While FIG. 2 depicts an illustrative embodiment of the ankle support brace frame 200, other equally effective ankle support brace frame configurations are also possible. For example, although the ankle support brace frame 200 is depicted in FIG. 2 as having a generally rectangular configuration using a flat or planar endwall member 206, in other embodiments, the ankle support brace frame 200 can have an outwardly arched or arcuate endwall member 206 to accommodate the generally arched shape of a human heel. Similarly, although the sidewall members 204 are depicted in FIG. 2 as generally planar, parallel, members, other sidewall member physical geometries are possible. For example, the sidewall members 204 may be disposed at a skew angle such that the distance between the sidewall members is narrowest at the closed-end of the ankle support brace frame 200 proximate endwall member 206 and widens toward the open-end of the frame to generally conform to the shape of a human foot. Such alternative configurations should be considered as falling within the scope of this disclosure.

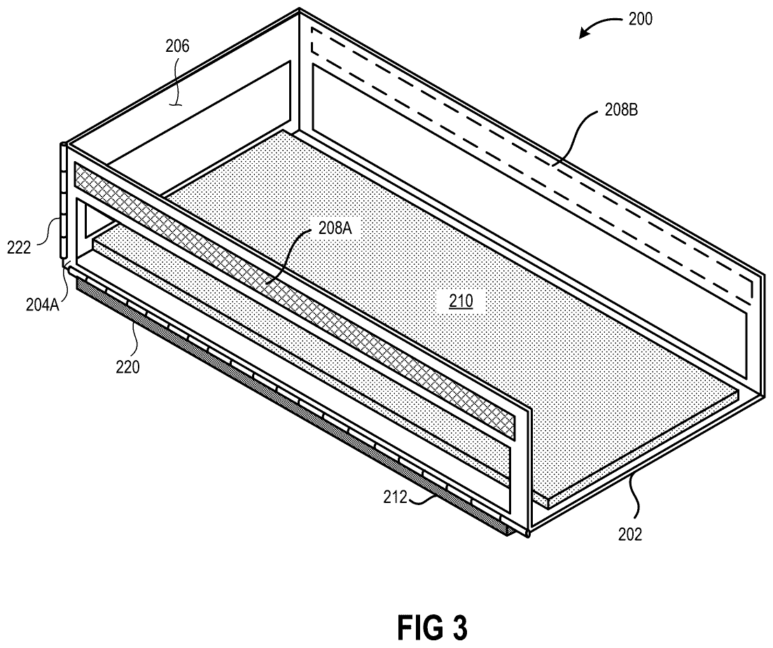

FIG. 3 is a perspective view of another illustrative ankle support brace frame 200 in accordance with one or more embodiments described herein. As depicted in FIG. 3, at least a portion of the sidewall members 204A and 204B include one or more open portions, to provide at least partially open sidewall members 204A and 204B. Similarly, the endwall member 206 may also include one or more open portions to provide an at least partially open endwall member 206.

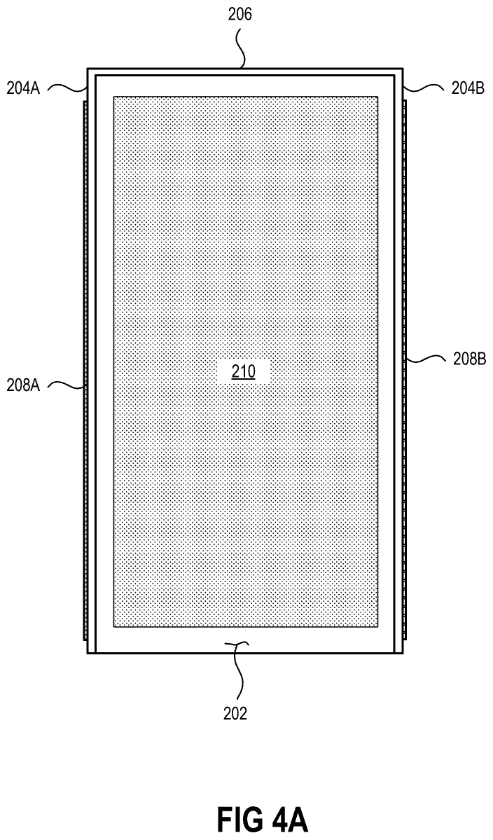

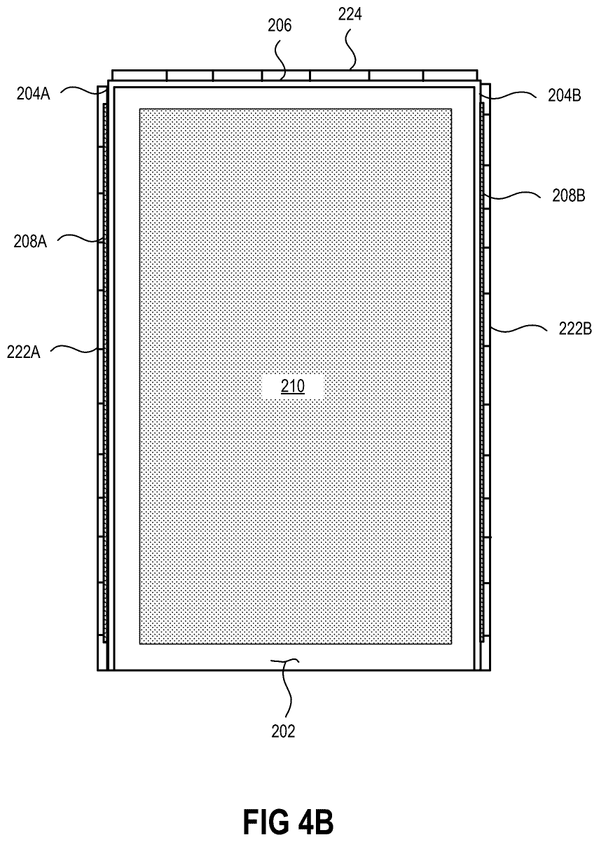

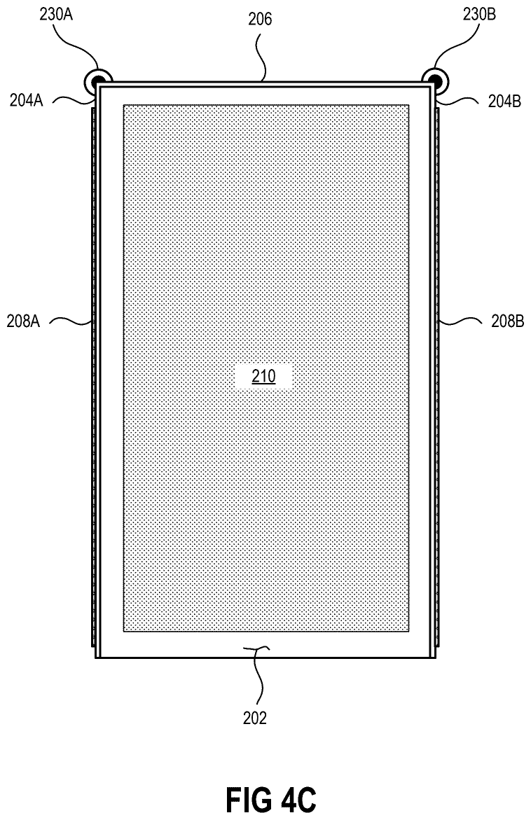

FIG. 4A is a top plan view of the illustrative ankle support brace frame 200 as depicted in FIG. 2, in accordance with one or more embodiments described herein. In some implementations, the interior surface feature 210 can include a uniform layer, coating, or surface treatment deposited, bonded, attached, laminated, formed, or otherwise disposed in, on, about, or across all or a portion of the interior surface of base member 202. In other implementations, all or a portion of the interior surface feature 210 can include a layer, coating, or surface treatment deposited, bonded, attached, laminated, formed, or otherwise disposed in a defined pattern, a random pattern, or a pseudo-random pattern in, on, about, or across all or a portion of the interior surface of base member 202. Functionally, the interior surface feature 210 provides a non-slip, non-skid, or similar surface treatment that resists or otherwise limits the slippage of the ankle support brace 100 when the brace is placed, positioned, or disposed proximate all or a portion of the interior surface feature 210.

FIG. 4B is a top plan view of another illustrative embodiment of the ankle support brace frame 200 depicted in FIG. 2 in which one or more hinges 222A and 222B (collectively, “hinges 222”) rotatably couple the sidewall members 204A and 204B, respectively, to the base member 202 and one or more hinges 224 rotatably couple the endwall member 206 to the base member 202, in accordance with one or more embodiments described herein. The hinges 222 can include but are not limited to one or more barrel hinges, piano hinges, spring loaded hinges, strap hinges, or similar. In some implementations, the hinges 222 may include a plurality of hinges placed or positioned at discrete locations and/or intervals along the joint between the lower edges of each of the sidewall members 204 and the side edges of the base member 202. In other implementations, the hinges 222 may include a single hinge, such as a piano hinge disposed along all or a portion of the joint between the lower edges of the sidewall members 204 and the side edges of the base member 202. In yet other implementations, the hinges 222 may include locking hinges that allow rotation of the sidewall members 204 to one or more defined angles (e.g., 90 degrees) measured with respect to the base member 202 whereupon the locking hinges 202 lock the sidewall members 204 in position. In yet other implementations, the hinges 222 may include spring loaded hinges that bias each of the sidewall members 204 to a defined angle (e.g., 90 degrees) measured with respect to the base member 202.

The one or more hinges 224 can include but are not limited to one or more barrel hinges, piano hinges, spring loaded hinges, strap hinges, or similar. In some implementations, the one or more hinges 224 may include a plurality of hinges placed or positioned at discrete locations along the joint between the lower edge of the endwall member 206 and edge of the base member 202. In other implementations, the one or more hinges 224 may include a single hinge, such as a piano hinge disposed along all or a portion of the joint between the lower edge of the endwall member 206 and the edge of the base member 202. In yet other implementations, the one or more hinges 224 may include locking hinges that allow rotation of the endwall member 206 to one or more defined angles (e.g., 90 degrees) measured with respect to the base member 202 whereupon the locking hinges 202 lock the endwall member 206 with respect to the base member 202. In yet other implementations, the one or more hinges 224 may include spring loaded hinges that bias the endwall member 206 to a defined angle (e.g., 90 degrees) measured with respect to the base member 202.

FIG. 4C is a top plan view of yet another illustrative embodiment of the ankle support brace frame 200 depicted in FIG. 2 in which one or more hinges 230A and 230B (collectively, “hinges 230”) rotatably couple each of the sidewall members 204A and 204B, respectively, to the endwall member 206, in accordance with one or more embodiments described herein. In some implementations, the hinges 230 may include a plurality of hinges placed or positioned at discrete locations along the joint between the rear edges of each of the sidewall members 204A and 204B and the side edges of the endwall member 206. In other implementations, the one or more hinges 230 may include a single hinge, such as a piano hinge disposed along all or a portion of the joint between the rear edges of each of the sidewall members 204A and 204B and the side edges of the endwall member 206. In yet other implementations, the one or more hinges 230 may include locking hinges that allow rotation of each of the sidewall members 204A and 204B to one or more defined angles (e.g., 90 degrees) measured with respect to the endwall member 206 whereupon the one or more locking hinges 230A and 230B lock the sidewall members 204A and 204B at the defined angle with respect to the endwall member 206. In yet other implementations, the one or more hinges 230 may include spring loaded hinges that bias each of the sidewall members 204 to a defined angle (e.g., 90 degrees) measured with respect to the endwall member 206. The use of one or more hinges as depicted in FIGS. 4B and 4C beneficially permit folding the ankle support brace frame 200 for transport and/or storage.

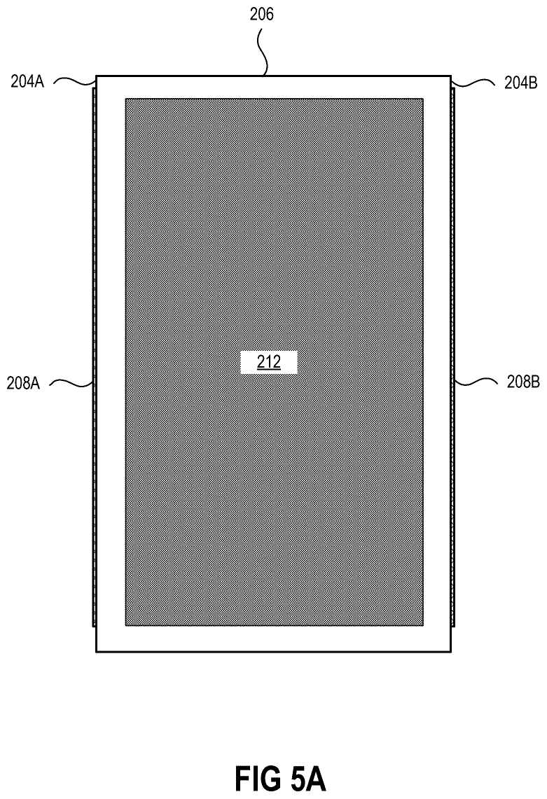

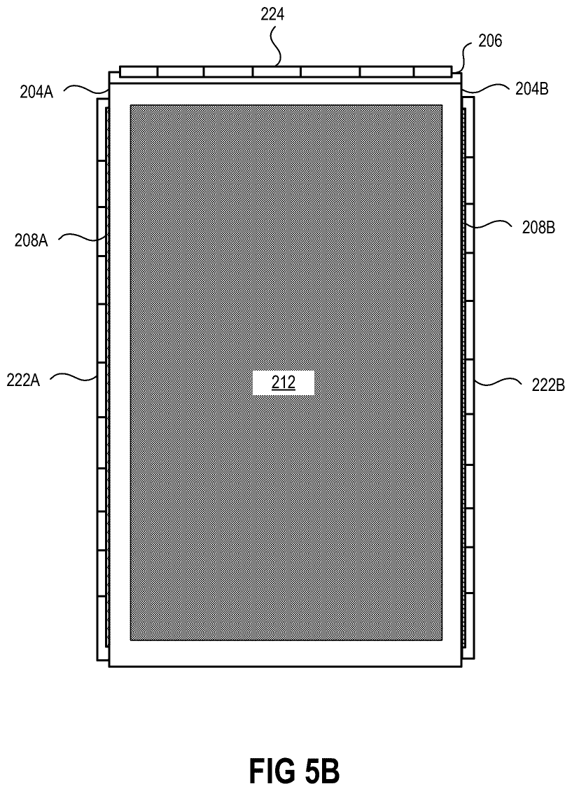

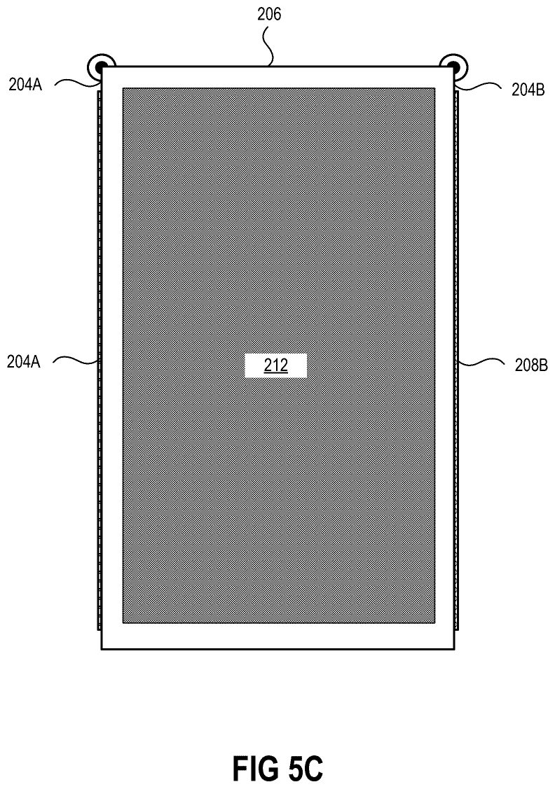

FIG. 5A is a bottom plan view of the ankle support brace frame 200 as depicted in FIG. 2, in accordance with one or more embodiments described herein. As depicted in FIG. 5A, in at least some implementations, the exterior surface feature 212 includes a uniform layer, coating, or surface treatment deposited, bonded, attached, laminated, formed, or otherwise disposed in, on, about, or across all or a portion of the exterior surface of base member 202. In other implementations, all or a portion of the exterior surface feature 212 includes a layer, coating, or surface treatment deposited, bonded, attached, laminated, formed, or otherwise disposed in a defined pattern, a random pattern, or a pseudo-random pattern in, on, about, or across all or a portion of the exterior surface of base member 202. The exterior surface feature 212 provides a non-slip, non-skid, or similar surface treatment that resists, impedes, or otherwise limits the slippage or movement of the ankle support brace frame 200 across an underlying support surface, particularly when a wearer inserts their foot into an opened ankle support brace 100 that has been disposed in the ankle support brace frame 200.

FIG. 5B is a bottom plan view of the illustrative embodiment of the ankle support brace frame 200 depicted in FIG. 4B in which the one or more hinges 222A and 222B (collectively, “hinges 222”) rotatably couple the sidewall members 204A and 204B, respectively, to the base member 202 and the one or more hinges 224 rotatably couple the endwall member 206 to the base member 202, in accordance with one or more embodiments described herein. FIG. 5C is a bottom plan view of the illustrative embodiment of the ankle support brace frame 200 depicted in FIG. 4C in which the one or more hinges 230A and 230B (collectively, “hinges 230”) rotatably couple each of the sidewall members 204A and 204B, respectively, to the endwall member 206, in accordance with one or more embodiments described herein.

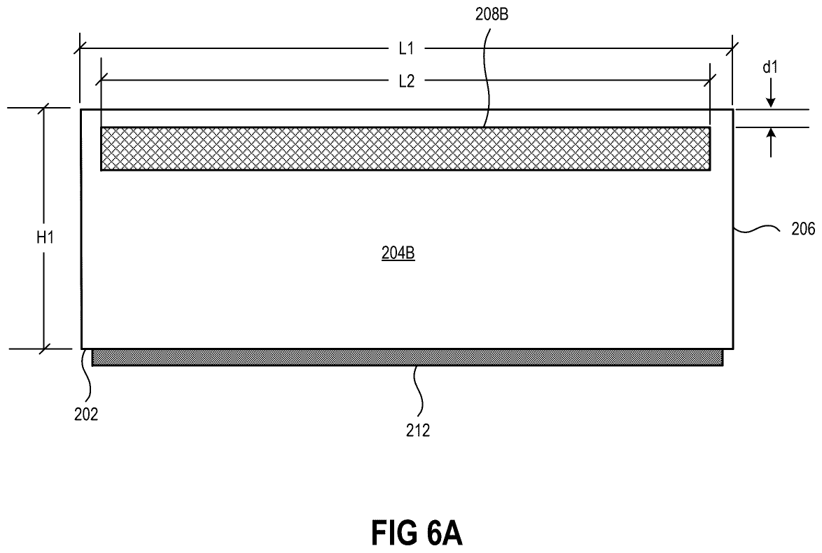

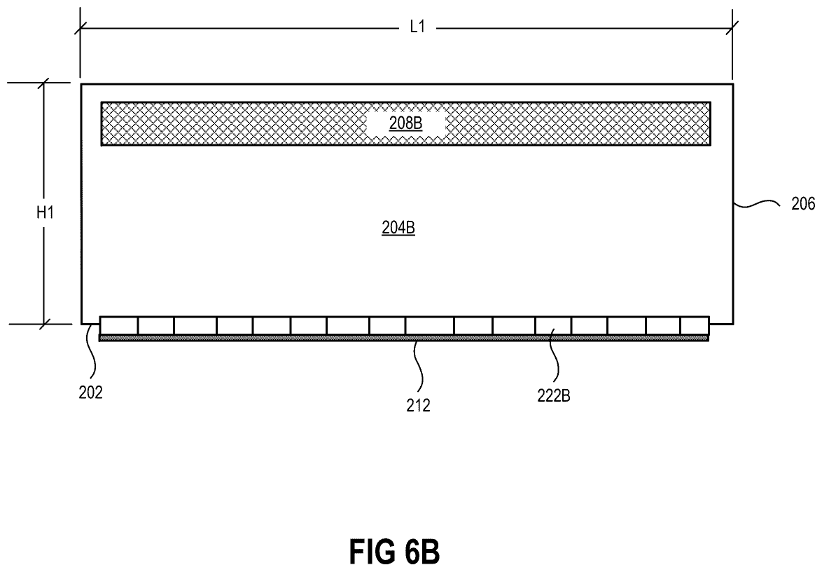

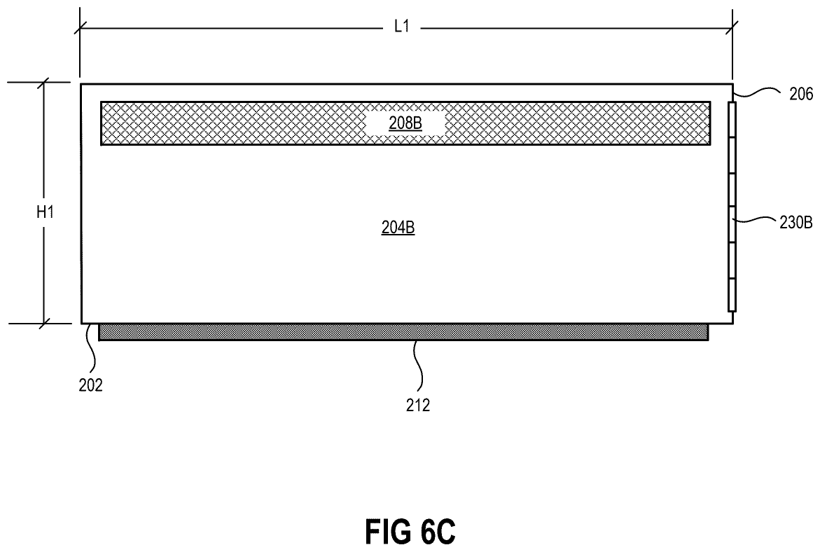

FIG. 6A is a side elevation of the ankle support brace frame 200 as depicted in FIG. 2, in accordance with one or more embodiments described herein. As depicted in FIG. 6A, sidewall member 204B can have an overall length “L1” and an overall height “H1.” Although discussed below in terms of sidewall member 204B, one should readily appreciate and understand that the features described below in the context of side member 204B are equally applicable to sidewall member 204A.

At least a portion of the exterior surface of sidewall member 204A includes attachment features 208A configured to capture, attach to, couple to, or otherwise engage at least a portion of the first attachment devices 104 of the ankle support brace 100. Similarly, at least a portion of the exterior surface of sidewall member 204B includes attachment features 208B configured to capture, attach to, couple to, or otherwise engage at least a portion of the second attachment devices 106 of the ankle support brace 100.

The attachment features 208B may be disposed or otherwise positioned at one or more locations on the exterior surface of the sidewall member 204B. For example, as depicted in FIG. 6A, the attachment features 208B may be disposed at, near, at a distance from, or proximate the upper edge of sidewall member 204B. In embodiments, the attachment features 208B may be disposed a distance “d1” measured from or relative to the upper edge of sidewall member 204B. The distance “d1” can be: about 0.25 inches or less, about 0.375 inches or less, about 0.50 inches or less, about 0.625 inches or less, about 0.75 inches or less, about 1 inch or less, about 1.25 inches or less, or about 1.5 inches or less. In some implementations, the attachment features 208B may include a unitary or single attachment feature 208B that extends along at least a portion of the length of the sidewall member 204B. For example, as depicted in FIG. 6A, a single attachment feature 208B can extend along the exterior surface of sidewall member 208B for a length “L2.” In such implementations the length “L2” is less than the length “L1” of the sidewall member 208B. In other implementations, the attachment features 208B may include a plurality of discrete attachment features disposed in a regular pattern, a random pattern, or a pseudo-random pattern in, on, or about the exterior surface of sidewall member 204B. In yet other implementations, the one or more attachment features 208B can include a single or unitary attachment feature that extends about the perimeter formed by sidewall members 204A and 204B and the endwall member 206 of the ankle support brace frame 200. At least a portion of the first attachment devices 104 attach to, couple to, or otherwise engage the attachment features 208A and at least a portion of the second attachment devices 106 attach to, couple to, or otherwise engage the attachment features 208B responsive to the ankle support brace 100 being draped, positioned, or placed over the sidewall members 204A and 204B and/or endwall member 206 of the ankle support brace frame 200.

FIG. 6B is a side elevation view of the illustrative embodiment of the ankle support brace frame 200 depicted in FIG. 4B in which the one or more hinges 222A and 222B (collectively, “hinges 222”) rotatably couple the sidewall members 204A and 204B, respectively, to the base member 202 and the one or more hinges 224 rotatably couple the endwall member 206 to the base member 202, in accordance with one or more embodiments described herein. As depicted in FIG. 6B, the exterior surface feature 212 disposed on the exterior surface of the base member 202 may extend beyond the hinges 222 such that when placed on a support surface, the exterior surface feature 212 contacts or rests proximate the underlying support surface.

FIG. 6C is a side elevation view of the illustrative embodiment of the ankle support brace frame 200 depicted in FIG. 4C in which the one or more hinges 230A and 230B (collectively, “hinges 230”) rotatably couple each of the sidewall members 204A and 204B, respectively, to the endwall member 206, in accordance with one or more embodiments described herein. As depicted and described above with respect to FIG. 6A, the rear-hinged sidewall member 204B includes one or more attachment features 208B disposed on at least a portion of the exterior surface of the rear-hinged sidewall member 204B.

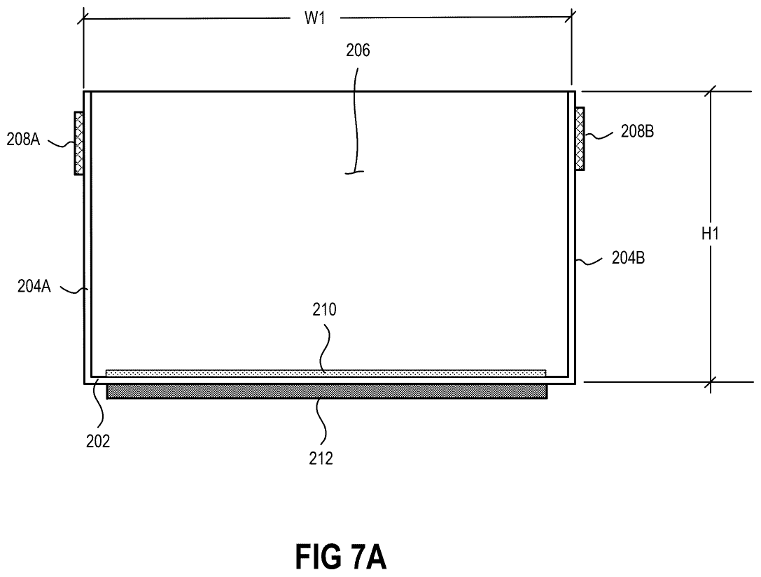

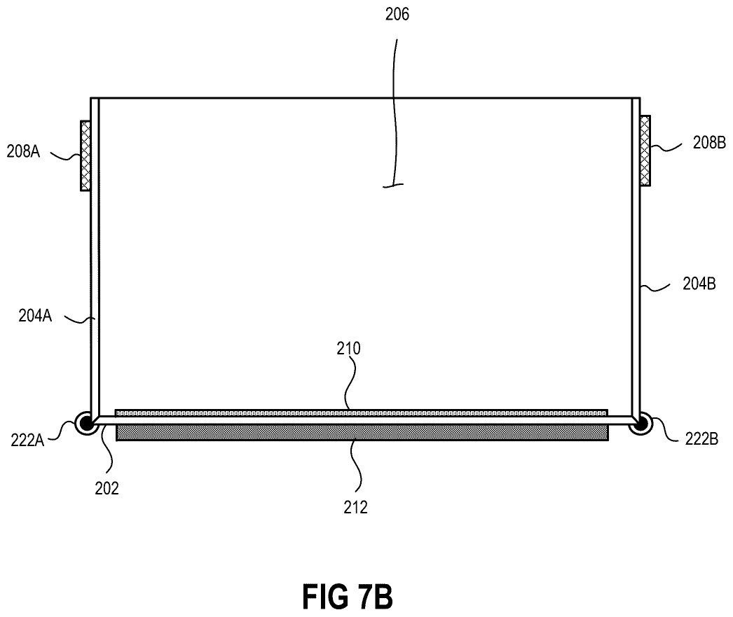

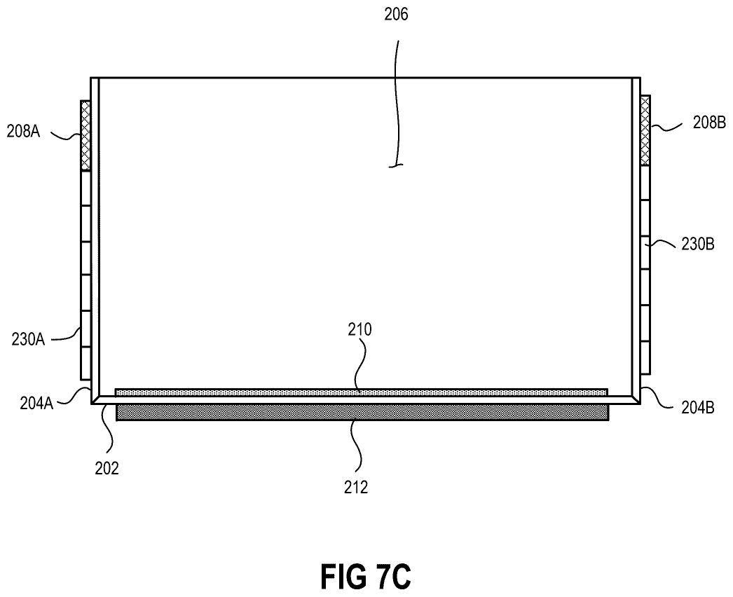

FIG. 7A is an open-end elevation view of the ankle support brace frame 200 depicted in FIG. 2, in accordance with one or more embodiments described herein. As depicted in FIG. 7A, the ankle support brace frame can have an overall height “H1” and an overall width “W1.” Attachment features 208 are positioned, attached, physically coupled to, or otherwise disposed on the exterior surface of each of the sidewall members 204. The attachment features 208 are configured to couple to or otherwise engage at least a portion of the first attachment devices 104 and/or at least a portion of the second attachment devices 106 of the ankle support brace 100. The attachment features 208 may be disposed or otherwise positioned at one or more locations on the exterior surface of each of the sidewall members 204. For example, as depicted in FIG. 7A, the attachment features 208 may be disposed at, near, proximate, or at a distance “d1” from the upper edge of the exterior surface of each of the sidewall members 204. The location of the attachment features 208 on the exterior surface of the sidewall members 204 is not limited to the location depicted in FIG. 7A—in other embodiments, the attachment features 208 may be positioned, located, or otherwise disposed at any location on the exterior surface of the sidewall members 204.

As depicted in FIG. 7A, the interior surface feature 210 may be deposited, bonded, attached, laminated, formed, or otherwise disposed in, on, about, or across all or a portion of the interior surface of the base member 202. Similarly, the exterior surface feature 212 may be deposited, bonded, attached, laminated, formed, or otherwise disposed in, on, about, or across all or a portion of the exterior surface of base member 202.

FIG. 7B is an open-end elevation view of the illustrative embodiment of the ankle support brace frame 200 depicted in FIG. 4B in which the hinges 222 rotatably couple the sidewall members 204 to the base member 202 and one or more hinges 224 rotatably couple the endwall member 206 to the base member 202, in accordance with one or more embodiments described herein. As depicted in FIG. 7B, the exterior surface feature 212 disposed on the exterior surface of the base member 202 may extend from beyond the hinges 222 such that when placed on a support surface, the exterior surface feature 212 limits or prevents the contact between the hinges 222 and the underlying support surface.

FIG. 7C is an open-end elevation view of the illustrative embodiment of the ankle support brace frame 200 depicted in FIG. 4C in which hinges 230 rotatably couple each of the sidewall members 204 to the endwall member 206, in accordance with one or more embodiments described herein.

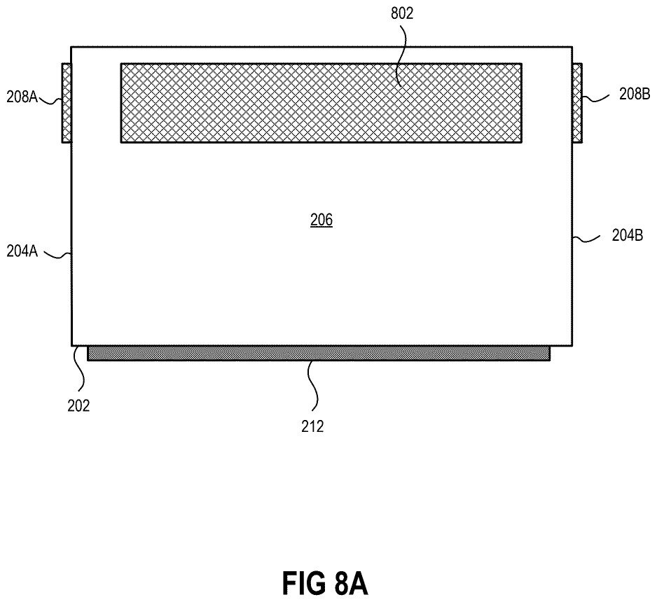

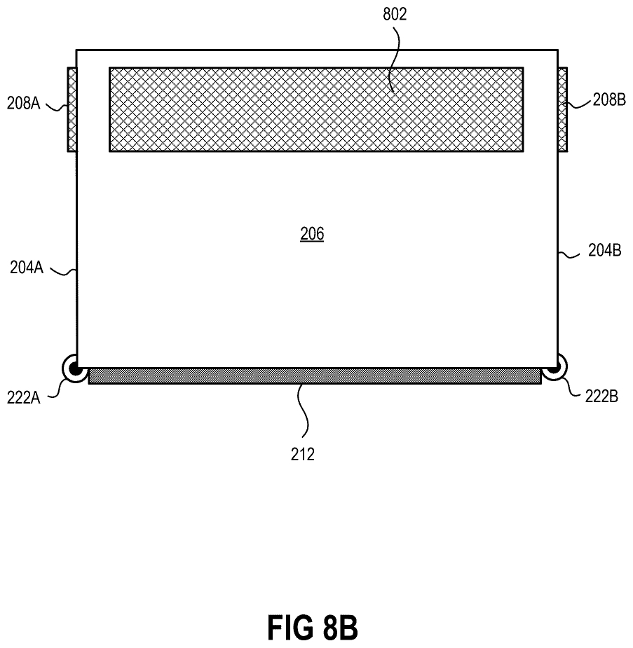

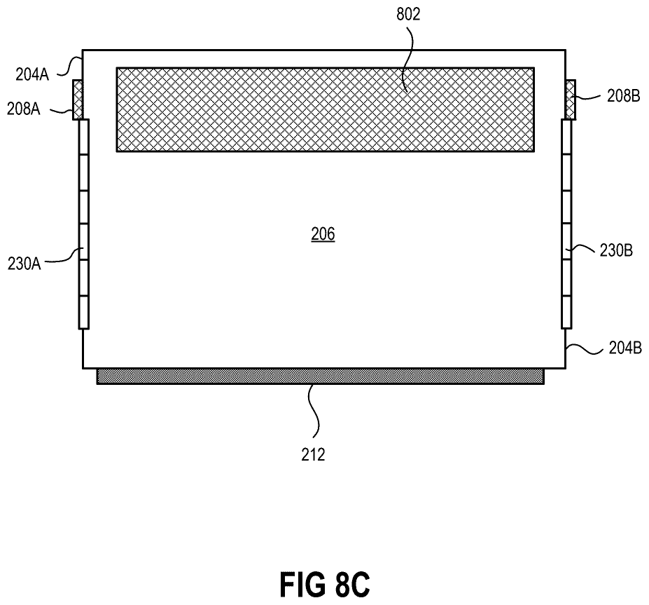

FIG. 8A is a closed-end elevation view of the ankle support brace frame 200 as depicted in FIG. 2, in accordance with one or more embodiments described herein. As depicted in FIG. 8A, in at least some embodiments one or more attachment features 802 can be disposed in, on, across, or about at least a portion of the exterior surface of the endwall member 206. The one or more attachment features 802 can include any system, device, or accessory capable of retaining the first attachment devices 104 and/or the second attachment devices 106 of the ankle support brace 100. In embodiments, the one or more attachment features 802 may include a hook-and-loop type material to retain at least a portion of the first attachment devices 104 and/or at least a portion of the second attachment devices 106. In other embodiments, the one or more attachment features 802 may include an elastic or resilient material coupled to the exterior surface of the endwall member 206. For example, the one or more attachment features 802 can include an elastic material such as rubber, nylon, or similar resilient materials to hold or otherwise retain at least a portion of the first attachment features 104 and/or at least a portion of the second attachment features 106 proximate, near, or against the exterior surface of the endwall member 206. In yet other embodiments, the one or more attachment features 802 may include a mechanical fastener such as a buckle, snap, quick-release, or similar device capable of retaining, coupling, physically attaching, or otherwise holding and releasing at least a portion of the first attachment devices 104 and/or at least a portion of the second attachment devices 106.

FIG. 8B is a closed-end elevation view of the illustrative embodiment of the ankle support brace frame 200 depicted in FIG. 4B in which the hinges 222 rotatably couple the sidewall members 204 to the base member 202 and one or more hinges 224 rotatably couple the endwall member 206 to the base member 202, in accordance with one or more embodiments described herein. As depicted and described above with respect to FIG. 8A, in at least some embodiments, the endwall member 206 may include one or more attachment features 802. As depicted in FIG. 8B, the exterior surface feature 212 disposed on the exterior surface of the base member 202 may extend from beyond the hinges 222 such that when placed on a support surface, the exterior surface feature 212 limits or prevents the contact between the hinges 222 and the underlying support surface.

FIG. 8C is a closed-end elevation view of the illustrative embodiment of the ankle support brace frame 200 depicted in FIG. 4C in which hinges 230 rotatably couple each of the sidewall members 204 to the endwall member 206, in accordance with one or more embodiments described herein.

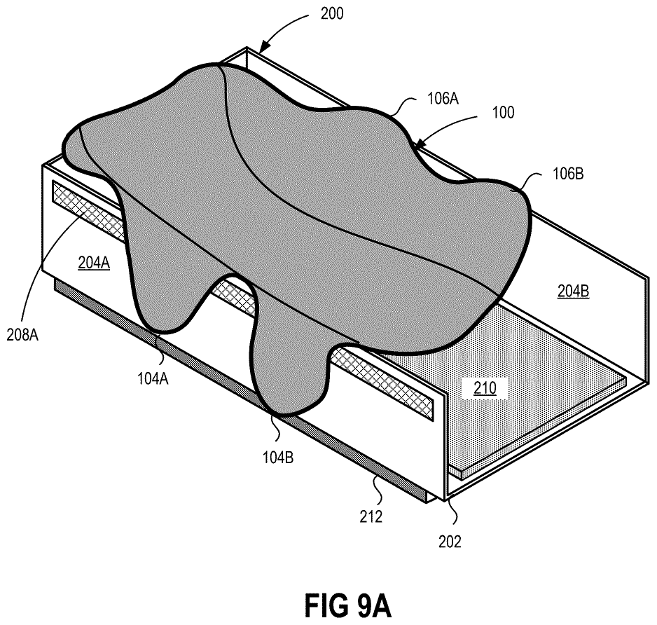

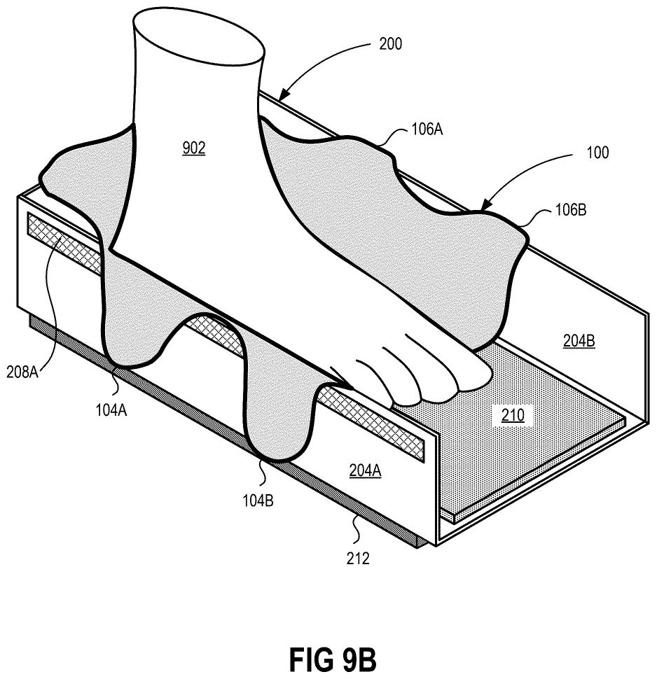

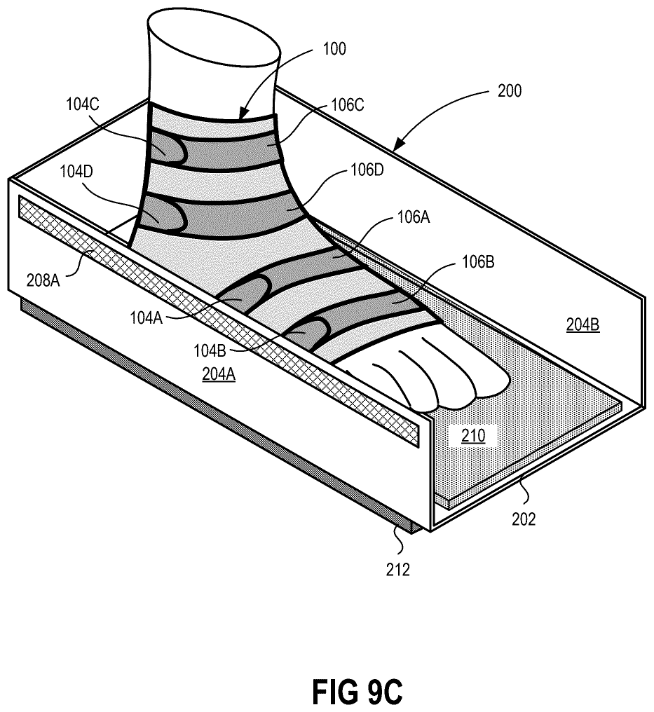

FIG. 9A is a perspective view of an illustrative ankle support brace 100 in an opened position disposed at least partially in the ankle support brace frame 200 as depicted in FIG. 2, in accordance with one or more embodiments described herein. As depicted in FIG. 9A, when the ankle support brace 100 is disposed or otherwise positioned inside the ankle support brace frame 200 preparatory to use, at least a portion of the first attachment devices 104A and 104B drape over the top edge of sidewall member 204A and physically attach, couple, or otherwise engage the one or more attachment features 208A disposed on the exterior surface of sidewall member 204A. Similarly, at least a portion of the one or more second attachment devices 106A and 106B drape over the top edge of sidewall member 204B and physically attach, couple, or otherwise engage the one or more attachment features 208B disposed on the exterior surface of sidewall member 204B. In at least some embodiments, the attachment features 208A and 208B may include a hook-and-loop type material to which the first attachment devices 104 and the second attachment devices 106 physically couple.

In other embodiments, the attachment features 208 may include an elastomeric or resilient material pulled taught such that the attachment features 208 remain at, on, near, or proximate the exterior surface of each of the sidewall members 204. In such embodiments, at least a portion of the first attachment devices 104 and at least a portion of the second attachment devices 106 may be passed under the one or more attachment features 208 such that the first attachment devices 104 and the second attachment devices 106 are trapped against the exterior surface of each of the sidewall members 204 by the attachment features 208. In yet other embodiments, the attachment features 208 may include one or more physical attachment devices, such as buttons, snaps, or similar mechanical fasteners that physically couple to complementary mechanical fasteners integrated into and/or disposed in, on, about each of some or all of the first attachment devices 104 and each of some or all of the second attachment devices 106.

By coupling the first attachment devices 104 to the attachment features 208A on the exterior surface of sidewall member 204A and the second attachment devices 106 to the attachment features 208B on the exterior surface of sidewall member 204B the installation of the ankle brace 100 is eased since the first attachment devices 104 and the second attachment devices 106 remain affixed, coupled, physically retained, or otherwise attached to the ankle support brace frame 200 and thus are substantially less likely to interfere, entangle, or attach to each other, the user's foot, or other proximate structures while installing the ankle brace 100. Thus, the use of the ankle support brace frame 200 beneficially eases the fitment of the ankle support brace 100 by individuals who may experience limited flexibility or mobility.

FIG. 9B is a perspective view of the illustrative ankle support brace 100 in the opened position disposed at least partially in the ankle support brace frame 200 depicted in FIG. 2, with a wearer's foot 902 positioned within the opened brace, in accordance with one or more embodiments described herein. The process of donning, fitting, or installing the ankle support brace 100 on the wearer's foot and/or ankle commences with the wearer positioning their foot 902 within the open ankle support brace 100, positioning the brace 100 between the sole and heel of the user's foot 902 and at least a portion of the interior surface feature 210. In at least some embodiments, the wearer can position their heel proximate, against, or near the endwall member 206 of the ankle support brace frame 200, thereby positioning or trapping the ankle support brace 100 between the wearer's heel 902 and the endwall member 206.

The interior surface feature 210 increases the friction between the interior surface of the base member 202 and the ankle support brace 100. The increased friction created by the interior surface feature 210 reduces the likelihood that the open ankle support brace 100 slides, moves, or is otherwise displaced forward out of the open end of ankle support brace frame 200 when the wearer's foot 902 contacts the brace. In a similar manner, the exterior surface feature 212 increases the friction between the exterior surface of the base member 202 and the underlying support surface (e.g., surface of the floor) upon which the ankle support brace frame 200 rests. The increased friction created by the exterior surface feature 212 assists in preventing the ankle support brace frame 200 from sliding across the support surface when the wearer places their foot 902 in the open ankle support brace 200.

FIG. 9C is a perspective view of the illustrative ankle support brace 100 in a closed or fitted position about the wearer's foot 902 while the wearer's foot and the illustrative ankle support brace are positioned in the ankle support brace frame 200 depicted in FIG. 2, in accordance with one or more embodiments described herein. As depicted in FIG. 9C, after positioning their foot 902 in the ankle brace 100 as depicted in FIG. 9B, the wearer detaches a first of the one or more first attachment devices 104A and a first of the one or more second attachment devices 106A and couples the detached first attachment devices 104A to the detached second attachment device 106A. The process is repeated for each of the remaining first attachment devices 104B-104n and each of the remaining second attachment devices 106B-106n to secure the ankle brace 100 about the user's foot and ankle. In some embodiments, the first attachment devices 104 and the second attachment devices 106 may be attached, detachably attached, coupled, or physically coupled together using hook-and-loop type fasteners. In other embodiments, the first attachment devices 104 and the second attachment devices 106 may be attached, detachably attached, coupled, or physically coupled together using one or more buckles or similar fasteners. In yet other embodiments, the first attachment devices 104 and the second attachment devices 106 may be attached, detachably attached, coupled, or physically coupled together using one or more physical couplers such as snaps, latching connectors, or similar. Once the ankle support brace 100 is fitted to the user's foot, the user can remove their foot from the ankle support brace frame 200. Beneficially, the absence of any type of attachment feature on the interior surfaces of the sidewall members 204A and 204B and the endwall member 206 permits the unrestricted removal of the user's foot from the ankle support brace frame 200.

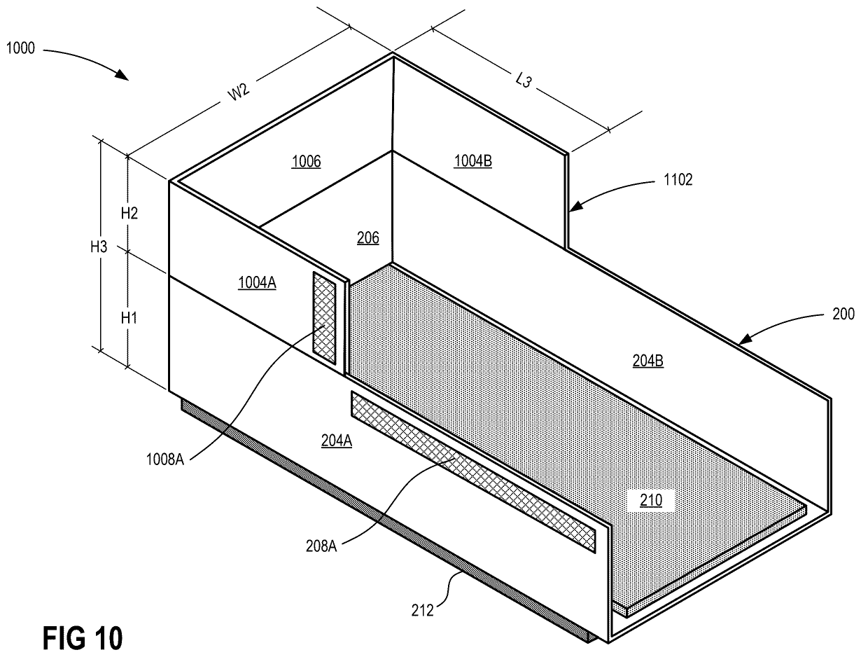

FIG. 10 is a perspective view of yet another embodiment of an ankle support brace frame 1000 in accordance with one or more embodiments described herein. As depicted in FIG. 10, the ankle support brace frame 1000 can include an upper ankle support brace frame 1002 disposed proximate at least a portion of the ankle support brace frame 200 as depicted in FIG. 2. The upper ankle support brace frame 1002 includes opposed upper sidewall members 1004A and 1004B (collectively, “upper sidewall members 1004”) connected by an upper endwall member 1006 to form an open channel-like structure having an interior surface and an exterior surface. As depicted in FIG. 10, the upper ankle support brace frame 1002 can have a length “L3”, width “W2”, and height “H2” suitable to accommodate the insertion of an upper portion of a wearer's ankle and/or the lower portion of a wearer's calf within the ankle brace support frame 1000. The upper ankle support brace frame 1002 includes one or more attachment features 1008A and 1008B (collectively, “attachment features 1008”) coupled, bonded, attached, or otherwise affixed to at least a portion of the exterior surface of upper sidewall members 1004A and 1004B, respectively. Although the upper ankle support brace frame 1002 is depicted in FIG. 10 as including a generally rectangular frame geometry, other geometries are possible. For example, the upper ankle support brace frame 1002 may include outwardly arched upper endwall member 1006 to accommodate the generally arched shape of the wearer's ankle and/or calf.

As depicted in FIG. 10, the upper ankle support brace frame 1002 is fitted to the top of a rear portion of the ankle support brace frame 200 such that sidewall member 204A of the ankle support brace frame 200 substantially aligns with upper sidewall member 1004A of the upper ankle support brace frame 1002. Similarly, sidewall member 204B of the ankle support brace frame 200 substantially aligns with the upper sidewall member 1004B of the upper ankle support brace frame 1002 and the endwall member 206 of the ankle support brace frame 200 substantially aligns with the upper endwall member 1006 of the upper ankle support brace frame 1002. In at least some implementations, the lower edge of the upper ankle support brace frame 1002 may be inserted at least partially within the ankle support brace frame 200.

In embodiments, the upper ankle support brace frame 1002 can include a unitary structure that includes the upper sidewall members 1004, and the upper endwall member 1006. In some embodiments, the upper ankle support brace frame 1002 can be fabricated using a metallic material such as aluminum, an aluminum alloy, or similar metal containing material. In other embodiments, the upper ankle support brace frame 1002 can be fabricated using a non-metallic material including, but not limited to: cardboard, injection molded plastic, carbon fiber, or similar rigid non-metallic materials. The upper ankle support brace frame 1002 can have any dimensions suitable for accommodating the insertion or placement of the ankle and/or lower calf within the upper ankle support brace frame 1000 and proximate the endwall member 1006 the upper ankle support brace frame 1002. The upper ankle support brace frame 1002 can have a length “L3” of: about 2 inches or less, about 3 inches or less; about 4 inches or less; or about 5 inches or less. The upper ankle support brace frame 1002 can have a height “H2” of: about 3 inches or less; about 4 inches or less; about 5 inches or less; or about 6 inches or less. The upper ankle support brace frame 1002 can have a width “W2” of: about 4 inches or less; about 6 inches or less; or about 8 inches or less.

Although FIG. 10 depicts the width “W2” of the upper ankle support brace frame 1002 as approximately equal to the width “W1” of the ankle support brace frame 200, the upper ankle support brace frame can have a width “W2” that is greater or lesser than the width “W1” of the ankle support brace frame 200. For example, in at least some embodiments, the upper ankle support brace frame 1002 may have a width “W2” less than the width “W1” of the ankle support brace frame 200 to enable the at least partial insertion of the upper ankle support brace frame 1002 into the ankle support brace frame 200. In other embodiments, the upper ankle support brace frame 1002 may have a width “W2” greater than the width “W1” of the ankle support brace frame 200 to enable the at least partial insertion of the upper ankle support brace frame 1002 into the ankle support brace frame 200.

The one or more attachment features 1008A and 1008B are disposed in, on, across, or about at least a portion of the exterior surface upper sidewall member 1004A and upper sidewall member 1004B, respectively. The one or more attachment features 1008 can include any system, device, or accessory capable of retaining the first attachment devices 104 and the second attachment devices 106 of the ankle support brace 100. In embodiments, each of the attachment features 1008 may include a hook-and-loop type material to which at least a portion of the first attachment devices 104 and at least a portion of the second attachment devices 106 can couple, bond, affix, or attach. In other embodiments, the attachment features 1008 may include an elastic or resilient material coupled to the exterior surface of each of the upper sidewall members 1004. For example, the attachment features 1008 can include an elastic material such as rubber, nylon, or similar resilient materials to hold or otherwise retain at least a portion of the first attachment devices 104 and at least a portion of the second attachment devices 106 proximate, near, adjacent, or against the exterior surface of the upper sidewall members 1004. In yet other embodiments, the attachment features 1008 may include a mechanical fastener such as a buckle, snap, quick-release, or similar device capable of retaining, coupling, physically attaching, or otherwise holding and releasing at least a portion of the first attachment devices 104 and at least a portion of the second attachment devices 106.

Although the upper ankle support brace frame 1002 is depicted in FIG. 10 as having solid upper sidewall members 1004 and a solid upper endwall member 1006, in embodiments, any or all of the upper sidewall members 1004 and/or the upper endwall member 1006 may include an open framework similar to the ankle support brace frame 200 as depicted in FIG. 3. Furthermore, although not depicted in FIG. 10, in embodiments one or more hinges disposed along the joints between the upper endwall member 1006 and the upper sidewall members to rotatably couple the upper sidewall members 1004 to the upper endwall member 1006.

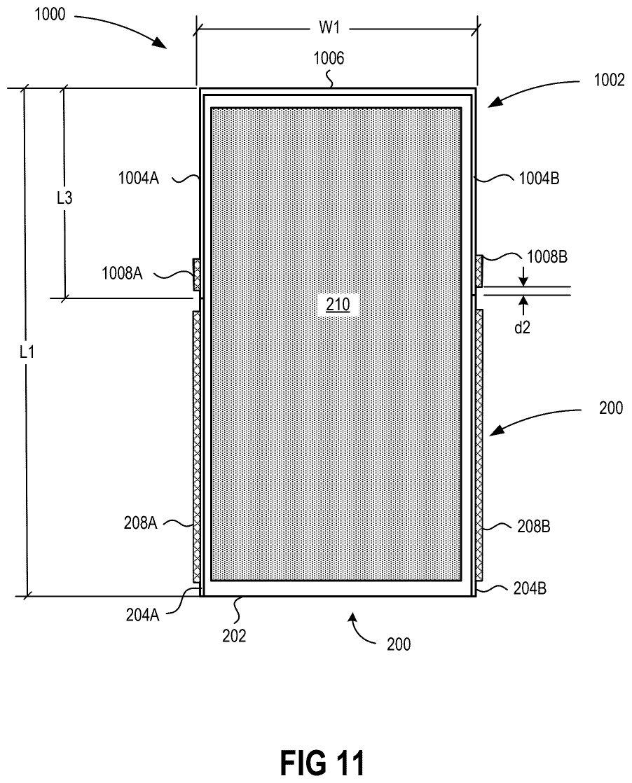

FIG. 11 is a top plan view of the ankle support brace frame 1000 depicted in FIG. 10, in accordance with one or more embodiments described herein. In some implementations. The upper ankle support brace frame 1002 is positioned proximate the top edge of the ankle support brace frame 200 as depicted in FIG. 2. Although the ankle support brace frame 200 and the upper ankle support brace frame 1002 have approximately the same width “W1” as depicted in FIG. 10, in some embodiments, the upper ankle support brace frame 1002 may have a different (i.e., smaller or larger) width “W2” than the width “W1” of the ankle support brace frame 200.

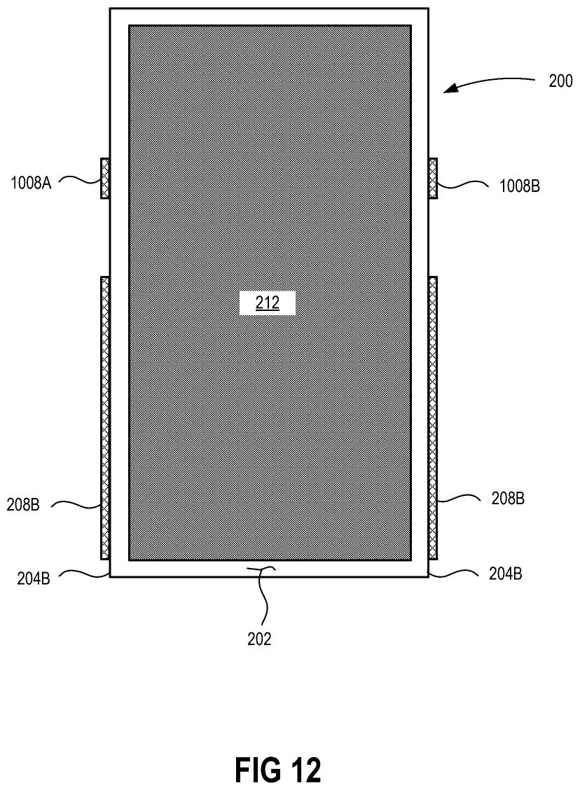

FIG. 12 is a bottom plan view of the upper ankle support brace frame 1000 as depicted in FIG. 10, in accordance with one or more embodiments described herein. As depicted in FIG. 12, in at least some implementations, the exterior surface feature 212 disposed in, on, about, or across all or a portion of the exterior surface of the base member 202 can include a uniform, continuous, layer, coating, or surface treatment deposited, bonded, attached, laminated, formed, or otherwise disposed in, on, about, or across all or a portion of the exterior surface of base member 202. In other implementations, all or a portion of the exterior surface feature 212 disposed in, on, about, or across all or a portion of the exterior surface of the base member 202 includes a layer, coating, or surface treatment deposited, bonded, attached, laminated, formed, or otherwise disposed in a defined pattern, a random pattern, or a pseudo-random pattern in, on, about, or across all or a portion of the exterior surface of base member 202.

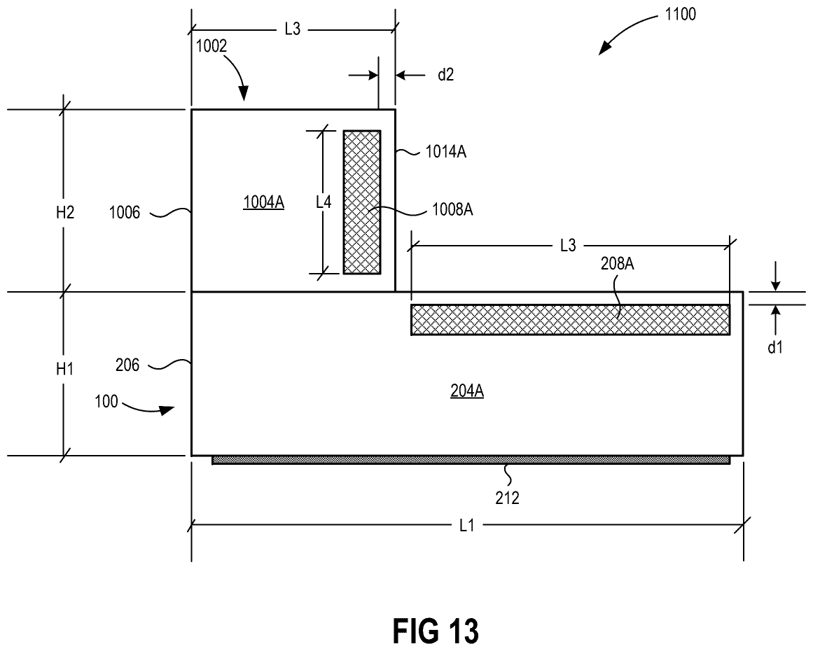

FIG. 13 is a side elevation of the ankle support brace frame 1000 as depicted in FIG. 10, in accordance with one or more embodiments described herein. As depicted in FIG. 13, the ankle support brace frame 1000 can include an ankle support brace frame 200 having sidewall members 208A and 204B with an overall length “L1” and a height “H1” and an upper ankle support brace frame 1002 having upper sidewall members 1004A and 1004B, each having a length “L3” and a height “H2.” The overall height “H3” of the ankle support brace frame 1000 can be the sum of height “H1” of ankle support brace 200 and height “H2” of upper ankle support brace height 1002. Although discussed below in terms of upper sidewall member 1004B, one should readily appreciate and understand that the features described below in the context of upper side member 1004B are equally applicable to upper sidewall member 1004A.

At least a portion of the exterior surface of upper sidewall member 1004A includes attachment features 1008A configured to capture, attach to, couple to, or otherwise engage at least a portion of the first attachment devices 104 of the ankle support brace 100. Similarly, at least a portion of the exterior surface of upper sidewall member 1004B includes attachment features 1008B configured to capture, attach to, couple to, or otherwise engage at least a portion of the second attachment devices 106 of the ankle support brace 100.

Each of the attachment features 1008A and 1008B may be disposed or otherwise positioned at one or more locations on the exterior surface of upper sidewall members 1004A and 1004B, respectively. In some embodiments, the attachment features 1008 may be disposed on the exterior surface of the sidewall members 1004 at a distance “d2” measured from the front or top edge of each of the sidewall members 1004. In various embodiments, the distance “d2” can be: about 0.25 inches or less, about 0.375 inches or less, about 0.50 inches or less, about 0.625 inches or less, about 0.75 inches or less, about 1 inch or less, about 1.25 inches or less, or about 1.5 inches or less.

For example, as depicted in FIG. 13, the attachment features 1008A and 1008B may be disposed at, near, or proximate the front edges of each of upper sidewall members 1004A and 1004B, respectively. In implementations, each of the attachment features 1008 may include a unitary attachment feature that extends along all or a portion of a length “L4” of the upper sidewall members 1004. As depicted in FIG. 13, the length “L4” is less than the height “H2” of the upper ankle support brace frame 1002. In other implementations, the one or more attachment features 1008 may include any number of separate or distinct attachment features 1008 disposed in a regular pattern, a random pattern, or a pseudo-random pattern on the exterior surface of each of the upper sidewall members 1004.

The one or more attachment features 1008 temporarily retain at least a portion of the first attachment devices 104 and/or at least a portion of the second attachment devices 106 of the ankle support brace 100. For example, responsive to the ankle support brace 100 being draped, positioned, folded, or otherwise disposed over the upper sidewall members 1004 of the upper ankle support brace frame 1002, the first attachment devices 104 and/or the second attachment devices 106 may couple to, contact, be retained by, or otherwise engage the attachment features 1008 disposed on the exterior surfaces of the upper side members 1004. The attachment features 1008 retain at least a portion of the first attachment devices 104 and/or at least a portion of the second attachment devices 106 when a wearer's ankle and/or calf is positioned proximate an open ankle support brace 100 disposed in the ankle support brace frame 1000.

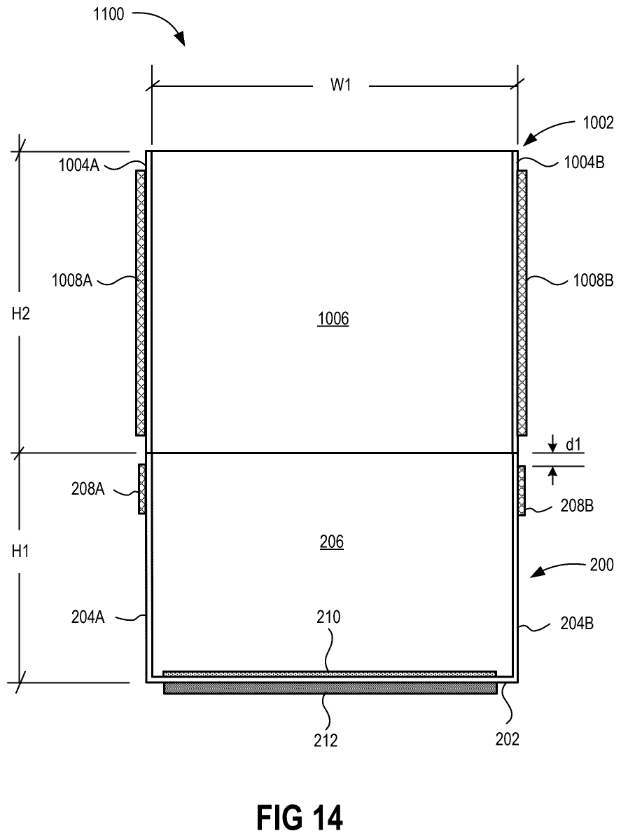

FIG. 14 is an open-end elevation view of the ankle support brace frame 1000 depicted in FIG. 10, in accordance with one or more embodiments described herein. As depicted in FIG. 14, the ankle support brace 200 includes attachment features 208 positioned, attached, physically coupled to, or otherwise disposed on the exterior surface of sidewall members 204. Further, as depicted in FIG. 14, the upper ankle support brace 1002 includes attachment features 1008 positioned, attached, physically coupled to, or otherwise disposed on the exterior surface of upper sidewall members 1004. The one or more attachment features 208 are configured to couple to or otherwise engage at least a portion of the first attachment devices 104 and at least a portion of the second attachment devices 106 of the ankle support brace 100. The one or more attachment features 1008 are configured to retain, attach to, couple to, or otherwise engage at least a portion of the first attachment devices 104 and at least a portion of the second attachment devices 106 of the ankle support brace 100.

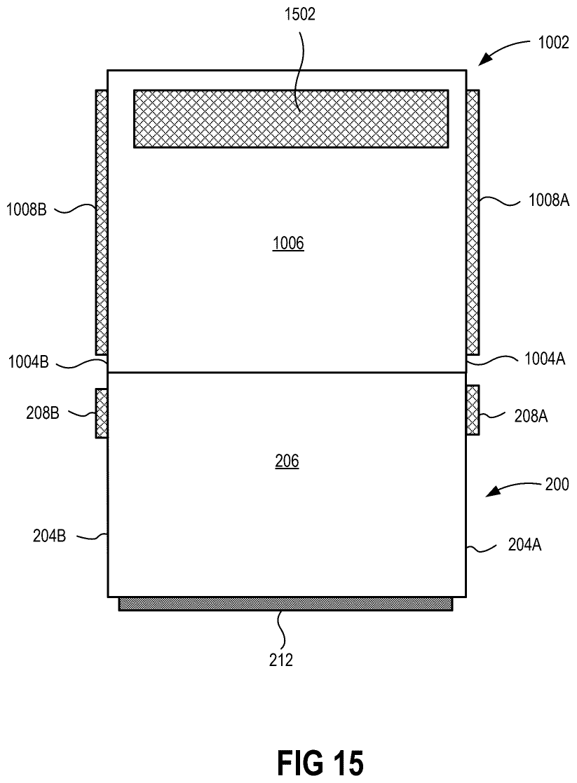

FIG. 15 is a closed-end elevation view of the ankle support brace frame 1000 depicted in FIG. 10, in accordance with one or more embodiments described herein. In embodiments, one or more attachment features1602 may be disposed in, on, across, or about at least a portion of the exterior surface of the upper endwall member 1006. The one or more attachment features 1602 can include any system, device, or accessory capable of retaining at least a portion of the ankle support brace 100, for example the central section 102 of the ankle support brace 100. In embodiments, the one or more attachment features 1602 may include a hook-and-loop type material to retain at least a portion of the ankle support brace 100. In other embodiments, the one or more attachment features 1602 may include an elastic or resilient material coupled to the exterior surface of the endwall member 1006. For example, the one or more attachment features 1602 can include an elastic material such as rubber, nylon, or similar resilient materials to hold, grasp, trap, or otherwise retain at least a portion of the ankle support brace 100 proximate, near, or against the exterior surface of the endwall member 1006. In yet other embodiments, the one or more attachment features 1602 may include a mechanical fastener such as a buckle, snap, quick-release, or similar device capable of retaining, coupling, physically attaching, or otherwise holding and releasing at least a portion of the ankle support brace 100.

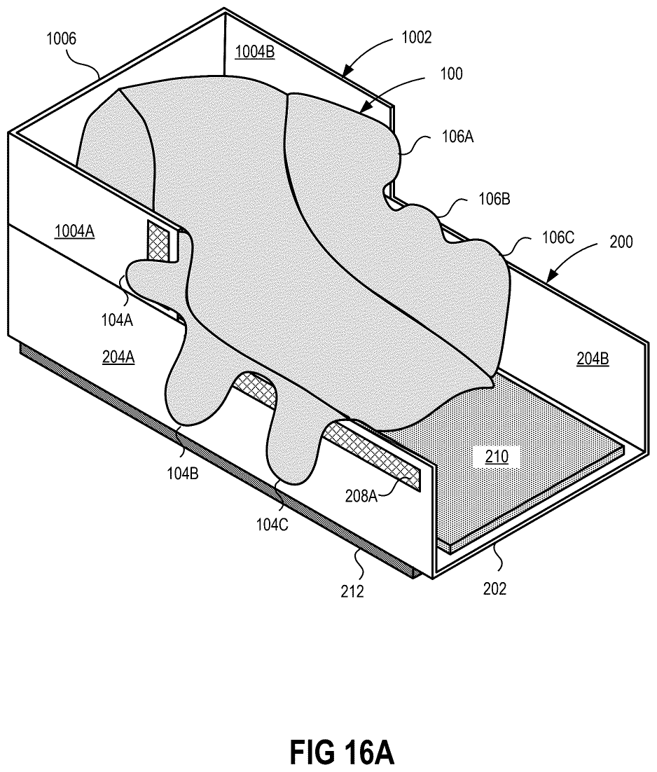

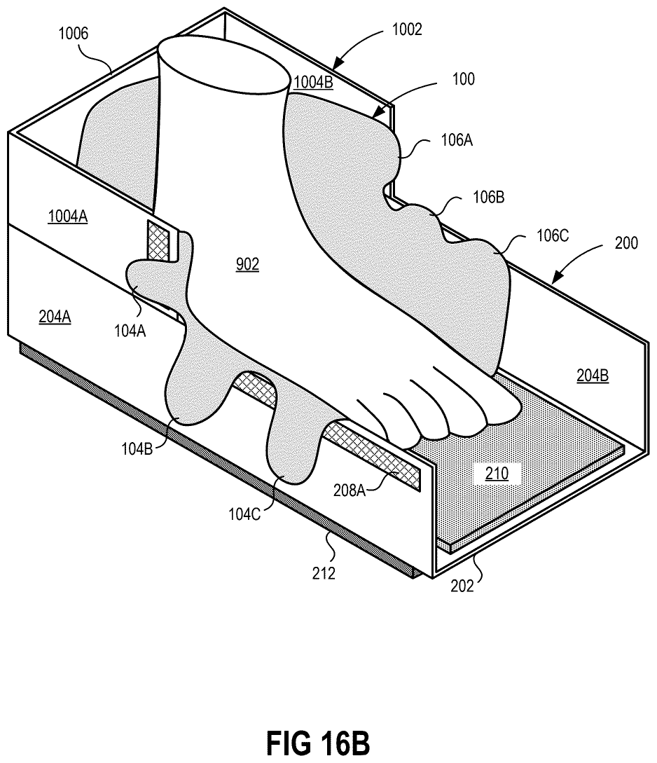

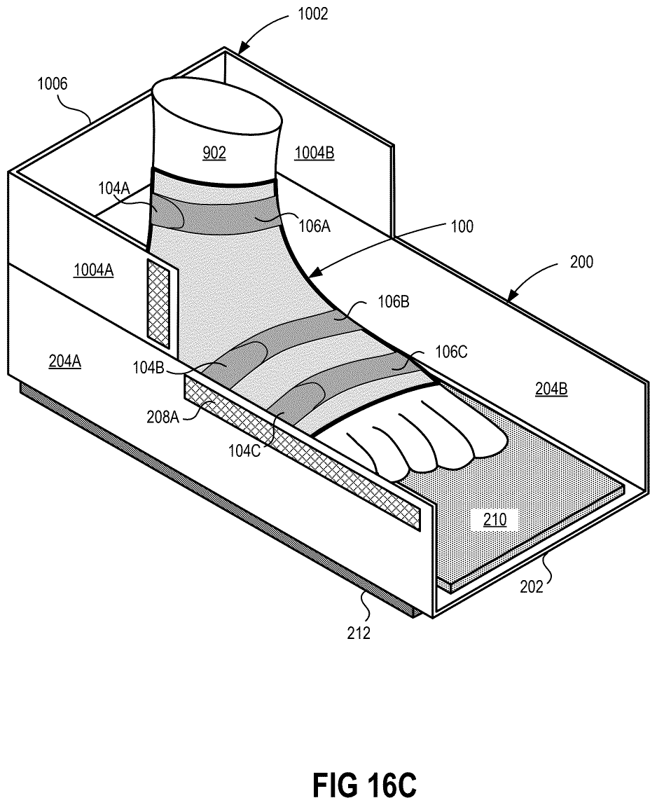

FIG. 16A is a perspective view of an illustrative ankle support brace 100 in an opened position disposed at least partially in the ankle support brace frame 1000 depicted in FIG. 10, in accordance with one or more embodiments described herein. As depicted in FIG. 16A, when the ankle support brace 100 is disposed or otherwise positioned inside the ankle support brace frame 1000 preparatory to use. Also as depicted in FIG. 16A, at least a portion of the first attachment devices 104 and at least a portion of the second attachment devices 106 drape over the top edge of the sidewall members 204 and around the front edge of the upper sidewall members 1004. At least a portion of the first attachment devices 104 and at least a portion of the second attachment devices 106 are physically coupled, retained, attached, or otherwise connected to the attachment features 208 disposed on the exterior surfaces of the sidewall members 208 and to the attachment features 1008 disposed on the exterior surfaces of the upper sidewall members 1008.

In at least some implementations, the attachment features 208 and the attachment features 1008 may include a hook-and-loop type material to which at least a portion of the first attachment devices 104 and at least a portion of the second attachment devices 106 detachably attach. In at least some implementations, the attachment features 208 and the attachment features 1008 may include a resilient or elastomeric material that can be stretched to accommodate the passage of the first attachment devices 104 and the second attachment devices 106 between the one or more attachment features 208 and 1008, the sidewall members 204 and the upper sidewall members 1004, respectively. In such implementations, the one or more attachment features 208 and 1008 may retain, hold, or otherwise maintain the first attachment devices 104 and the second attachment devices 106 proximate, near, against, or touching the exterior surfaces of the sidewall members 204 and upper sidewall members 1004, respectively, until the first attachment devices 104 and the second attachment devices 106 are detached from the attachment features 208 and 1008 by the wearer of the ankle support brace 100.

Coupling at least a portion of the first attachment devices 104 and at least a portion of the second attachment devices 106 to the one or more attachment features 208 and 1008 eases the installation of the ankle support brace 100 since the first attachment devices 104 and the second attachment devices 106 remain affixed, coupled, bonded, retained, or otherwise attached to ankle support brace frame 1000 and thus are substantially less likely to interfere, entangle, or attach to each other, the user's foot, or other proximate structures while installing the ankle support brace 100. Thus, the use of the ankle support brace frame 1000 beneficially eases the installation of the ankle support brace 100 by individuals experiencing limited flexibility or mobility.

FIG. 16B is a perspective view of the illustrative ankle support brace 100 in the opened position disposed at least partially in the ankle support brace frame 1000 depicted in FIG. 10, with a wearer's foot 902 positioned within the opened brace, in accordance with one or more embodiments described herein. The process of fitting or installing the ankle support brace 100 on the foot and ankle of a wearer commences with the user positioning their foot 902 within the open ankle support brace 100, which positions the open ankle support brace 100 between the wearer's foot, ankle, and lower calf and the interior surfaces of the ankle brace support frame 1000.

FIG. 16C is a perspective view of the illustrative ankle support brace 100 in a closed or fitted position about the wearer's foot 902 while the wearer's foot and the illustrative ankle support brace 100 are positioned in the ankle support brace frame 1000 depicted in FIG. 10, in accordance with one or more embodiments described herein. As depicted in FIG. 16C, after positioning their foot, ankle, and/or calf 902 in the ankle brace 1000 as depicted in FIG. 16B, the wearer detaches a first of the first attachment devices 104A and a first of the second attachment devices 106A and couples the detached first attachment devices 104A to the detached second attachment device 106A. The process is repeated for each of the remaining first attachment devices 104B-104n and each of the remaining second attachment devices 106B-106n to secure the ankle support brace 1000 about the wearer's foot and ankle.

Once the ankle support brace 1000 is fitted to the user's foot and/or ankle 902, the wearer can remove their foot, ankle, and/or calf from the ankle support brace frame 1000. The absence of any type of attachment feature on the interior surfaces of the sidewall members 204, the upper sidewall members 1004, the endwall member 206, and the upper endwall member 1006 beneficially permits the unrestricted removal of the wearer's foot, ankle, and/or calf from the ankle support brace frame 1000.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

We claim:

1. A brace support frame comprising:
a base member having an interior surface and an exterior surface;
wherein at least a portion of the interior surface of the base member includes an interior surface feature; and
wherein at least a portion of the exterior surface of the base member includes an exterior surface feature;
a first sidewall member having an interior surface and an exterior surface;
wherein at least a portion of the exterior surface of the first sidewall member includes one or more first attachment features;
a second sidewall member having an interior surface and an exterior surface;
wherein at least a portion of the exterior surface of the second sidewall member includes one or more second attachment features;
an endwall member having an interior surface and an exterior surface;
wherein the base member, the first sidewall member, the second sidewall member, and the endwall member form a channel having an open first end and a closed second end, the channel to receive an orthosis having one or more first attachment devices and one or more second attachment devices;
wherein the one or more first attachment features to couple to at least one of the one or more first attachment devices; and
wherein the one or more second attachment features to couple to at least one of the one or more second attachment devices.

2. The brace support frame of claim 1 wherein at least a portion of the exterior surface of the endwall member includes an exterior surface feature.

3. The brace support frame of claim 1 wherein the coefficient of static friction between the interior surface feature of the base member and the orthosis exceeds the coefficient of static friction between the interior surface of the base member and the orthosis.

4. The brace support frame of claim 3 wherein the interior surface feature of the base member creates a minimum coefficient of static friction of 0.5 between the interior surface feature of the base member and the orthosis.

5. The brace support frame of claim 3 wherein the coefficient of static friction between the exterior surface feature of the base member and an underlying support surface exceeds the coefficient of static friction between the exterior surface of the base member and the underlying support surface.

6. The brace support frame of claim 5 wherein the exterior surface feature of the base member creates a minimum coefficient of static friction of 0.5 between the exterior surface feature and an underlying support surface.

7. The brace support from of claim 4 wherein the one or more first attachment features comprise a hook-and-loop material affixed to the exterior surface of the first 2024 member.

8. The brace support frame of claim 5 wherein the one or more second attachment features comprise a hook-and-loop material affixed to the exterior surface of the second sidewall member.

9. The brace support frame of claim 1 wherein the endwall member comprises an arched endwall member.

10. The brace support frame of claim 1, further comprising:
one or more first hinges to rotatably couple a lower edge of the first sidewall member to a first edge of the base member; and
one or more second hinges to rotatably couple a lower edge of the second sidewall member to a second edge of the base member, the second edge of the base member transversely opposed across a width of the base member from the first edge of the base member.

11. The brace support frame of claim 8 further comprising:
one or more third hinges to rotatably couple a lower edge of the endwall member to a third edge of the base member, the third edge along the width of the base member.

12. The brace support frame of claim 1 further comprising:
one or more first hinges to rotatably couple a rear edge of the first sidewall member to a first edge of the endwall member; and
one or more second hinges to rotatably couple a rear edge of the second sidewall member to a second edge of the endwall member, the second edge of the endwall member transversely opposed across a width of the endwall member from the first edge of the endwall member.

13. The frame of claim 1, further comprising an upper brace support frame that includes:
an upper first sidewall member affixable to the first sidewall member, the upper first sidewall member to extend the height of a portion of the first sidewall member from a first height to a second height;
wherein the upper first sidewall member includes an interior surface and an exterior surface;
wherein at least a portion of the exterior surface of the upper first sidewall member includes one or more first upper attachment features; and
wherein the one or more first upper attachment features to couple to at least one of the one or more first attachment devices; and
an upper second sidewall member affixable to the second sidewall member, the upper second sidewall member to extend the height of a portion of the second sidewall member from the first height to the second height;
wherein the upper second sidewall member includes an interior surface and an exterior surface; and
wherein at least a portion of the exterior surface of the upper first sidewall member includes one or more second upper attachment features; and
wherein the one or more second upper attachment features to couple to respective ones of the one or more second attachment devices.
an upper endwall member affixable to the endwall member, the upper endwall member to extend the height of the endwall member from the first height to the second height;
wherein the upper endwall member includes an interior surface and an exterior surface.

14. The brace support frame of claim 13 wherein at least a portion of the exterior surface of the upper endwall member includes an exterior surface feature.

15. The brace support frame of claim 13 wherein the one or more first upper attachment features comprise a hook-and-loop material affixed to the exterior surface of the first upper sidewall member.

16. The brace support frame of claim 15 wherein the one or more second upper attachment features comprise a hook-and-loop material affixed to the exterior surface of the second upper sidewall member.

17. The brace support frame of claim 16 wherein the exterior surface of the upper endwall member includes one or more upper endwall attachment features.

18. The brace support frame of claim 17 wherein the one or more upper endwall attachment features comprise a hook-and-loop material affixed to the exterior surface of the upper endwall member.

19. The brace support frame of claim 13 wherein the upper endwall member comprises an arched upper endwall member.
